(19) United States
(12) Patent Application Publication
(10) Pub. No.: US 2010/0021556 A1
(43) Pub. Date: Jan. 28, 2010

(54) Method for the Production of an Agent Against an Infectious Disease

(76) Inventor: Rupert Temper, BgB (AT)

Correspondence Address: BACHMAN & LAPOINTE, P.C., 900 CHAPEL STREET, SUITE 1201, NEW HAVEN, CT, 06510, US

(21) Appl. No.: 12/513,674

(22) PCT Filed: Oct. 31, 2007
(86) PCT No.: PCT/EP2007/009490

§ 371 Date: Aug. 6, 2009

(30) Foreign Application Priority Data
Nov. 6, 2006 (DE) 10 2006 052 504.3

(51) Int. Cl. A61K 36/00; A61K 33/42; A61K 36/07; A61K 36/38; A61K 36/02; A61K 36/71; A61P 31/12

(52) U.S. Cl. 424/601; 424/725; 424/195.15; 424/730; 424/195.17

(57) ABSTRACT

A method for producing an agent against an infectious disease, particularly HIV, Ebola, or similar. In the method, a pressurized, especially medical oxygen is swirled into a solution containing at least one plant component particularly in the form of an extract.

### BACKGROUND OF THE INVENTION

The invention relates to a method for producing a composition against an infectious disease, in particular against HIV, Ebola or the like.

The treatment of HIV-infected people is one of the most urgent biomedical problems of recent times. It is as yet possible only to avoid an infection with the HIV virus by suitable measures, for example by using condoms during sexual intercourse. Once the HIV virus is present in the body, it is possible only to inhibit its effect and spread. Novel, promising therapies therefore relate to the inhibition of the rapid proliferation of the virus in human tissue. HIV prothease inhibitors block an important enzymatic metabolic pathway in the virus, leading to considerably reduced viral loads, thus slowing down the unremitting destruction of the immune system and the harmful effects, resulting therefrom, on human health.

A large number of chemical agents used for HIV injection treatment are known from the literature. These include for example azido derivatives of β-L-2′-nucleosides as disclosed in DE 699 30 378 C2. DE 600 06 706 C2 describes N-acrylmethylthioamilite derivatives for inhibiting HIV replication. DE 602 04 967 T2 describes oversulfated polysaccharides as HIV inhibitors. All these chemical agents have undesired side effects which are to be avoided.

DE 693 27 236 T2 describes the use of dietetic whey proteins for the treatment of HIV-seropositive individuals. In this case, a denatured whey protein concentrate is described for the production of a medicament for the treatment of these individuals. The concentrate is to be designed so that the T-helper cell concentrations and the T-helper cell/T-suppressor cell ratio in an HIV-seropositive individual is increased.

The problem of the present invention is to provide a method and composition which serve to control infectious diseases, in particular HIV, Ebola or the like and show few side effects.

### SUMMARY OF THE INVENTION

In accordance with the invention, medicinal oxygen is turbulently introduced under pressure into a solution which contains at least one plant constituent, in particular in the form of an extract, leading to the solution to the problem.

### DETAILED DESCRIPTION

Medicinal oxygen is used for example in artificial respiration and in inhalation therapies. For this purpose, oxygen must be subjected to a special preliminary process in which this oxygen is specially purified and its aggressive effect is reduced.

In the present method, the medicinal oxygen is turbulently introduced over the course of about one hour with a superatmospheric pressure of about two atmospheres into the solution in such a way that the maximum amount of oxygen is introduced into the solution and also remains in the solution.

The solution preferably used is a physiological magnesium phosphoricum solution. However, this is to be understood as only exemplary, and other solutions are conceivable.

In a first exemplary embodiment of the invention, an extract from Afacimmune is to be used in the solution. Afacimmune means the fungus Agaricus Campestris which is normally grown on mineralized compost soil.

In a second exemplary embodiment of the invention, elder bark/flowers and/or Agaricus Blazei Murill is used as extract in the solution. The latter is the so-called almond fungus which originally comes from the Brazilian rainforest. Scarcely any fungus stimulates the immune system as effectively as the Agaricus. Its content of polysaccharides, especially of beta-glucans, are the highest by comparison with other medicinal fungi. For this reason, it is used for cancers. Its promoting effect on the production of blood in the bone marrow is also known. It is also suitable for use for alleviating liver disorders and assists the spleen in its purification of blood and defense functions.

In a further exemplary embodiment of the invention, the extract consists of St. John's wort and/or parsley juice in the solution. An extract of blue algae and/or buttercup also appears to be particularly effective. The blue algae extract is to contain about 80 g of lithium per gram of dry matter.

The buttercup extract is produced by pouring hot triple-distilled water over carefully dried buttercups and leaving the mixture to extract for seven minutes, with the above-mentioned medicinal oxygen being turbulently introduced in particular into the buttercup extract.

In a further preferred exemplary embodiment of the invention, a sugar is admixed with the solution apart from the solution with the Afacimmune extract. It is possible in this case for the sugar to have been specially treated, but normal granulated sugar is also possible.

The respective extract is preferably produced with hot triple-distilled water. The latter is water which has been distilled three times and is of very high purity.

Protection is also sought for the corresponding products produced by the above-mentioned methods.

1-15. (canceled)

16. A method for producing a medicinal composition comprising the steps of:
providing a solution comprising an extract of at least one plant constituent; and
introducing into the solution medicinal oxygen under pressure to provide turbulence in the solution.

17. The method as claimed in claim 16, including introducing the medicinal oxygen over a period of about one hour under a superatmospheric pressure of 2 atmospheres to provide turbulence in the solution.

18. The method as claimed in claim 17, including providing a physiological magnesium phosphoricum solution.

19. The method as claimed in claim 16, wherein the extract is an extract from Afacimmune.

20. The method as claimed in claim 16, wherein the extract is an extract of at least one of (a) elder bark/flowers and (b) Agaricus Blazei Murill.

21. The method as claimed in claim 16, wherein the extract is an extract of at least one of (a) St. John's wort and (b) parsley juice.

22. The method as claimed in claim 16, wherein the extract is an extract of at least one of (a) blue algae and (b) buttercup.

23. The method as claimed in claim 22, wherein the blue algae extract contains 80 g of lithium per gram of dry matter.

24. The method as claimed in claim 22, wherein the buttercup extract is produced by pouring hot triple-distilled water over dried buttercups and leaving the mixture to extract for 7 minutes.

25. The method as claimed in claim 16, wherein sugar is added to the solution.

26. The method as claimed in claim 16, wherein the extract is produced with hot triple-distilled water.

27. A medicinal composition comprising sugar, elder bark/flower extract, and Agaricus Blazei Murill extract.

28. A medicinal composition comprising an extract of Afacimmune.

29. A medicinal composition comprising sugar, parsley juice, and St. John's wort extract.

30. A medicinal composition comprising sugar, blue algae extract, and buttercup extract.
